(19) United States
(12) Patent Application Publication
(10) Pub. No.: US 2024/0130631 A1
(43) Pub. Date: Apr. 25, 2024

(54) METHOD, DEVICE AND COMPUTER SYSTEM FOR OBTAINING RESPIRATORY SIGNAL

(71) Applicant: SOUTH CHINA NORMAL UNIVERSITY, Guangzhou (CN)

(72) Inventors: Baoxian YU, Guangzhou (CN); Yue HOU, Guangzhou (CN); Han ZHANG, Guangzhou (CN); Zhiqiang PANG, Guangzhou (CN)

(21) Appl. No.: 18/515,040

(22) Filed: Nov. 20, 2023

Related U.S. Application Data
(63) Continuation-in-part of application No. PCT/CN2022/136219, filed on Dec. 2, 2022.

(30) Foreign Application Priority Data
Jan. 10, 2022 (CN) 202210019272.0

(51) Int. Cl. A61B 5/08; A61B 5/00

(52) U.S. Cl. CPC A61B 5/0816; A61B 5/7207; A61B 5/725; A61B 5/7257

(57) ABSTRACT

Disclosed are a method, device, computer system for obtaining respiratory signal. The method includes: filtering aliasing vital signs signals obtained by piezoelectric sensor to obtain target vital signs signals; performing a Fourier transform to obtain first frequency response, and generating an upper envelope according to each frequency point of the first frequency response; determining main peak frequency point and main peak amplitude according to the frequency point corresponding to the maximum value of flat tops; identifying the flat top corresponding to the main peak amplitude as main peak flat top; determining minimum value frequency point according to the minimum value between the main peak flat top and an adjacent flat top or flat bottom; and determining respiratory spectrum principal component interval according to the minimum value frequency point; reconstructing the respiratory spectrum principal component interval through an empirical wavelet function to obtain reconstructed respiratory signal.

### CROSS-REFERENCE TO RELATED APPLICATIONS

This application claims priority to PCT Application No. PCT/CN2022/136219 filed on Dec. 2, 2022, which claims priority to Chinese Patent Application No. 202210019272.0 filed on Jan. 10, 2022, the entire contents of both of which are hereby incorporated by reference.

### FIELD OF TECHNOLOGY

The following relates to the technical field of respiratory signal, in particular to a method, device and computer system for obtaining respiratory signal.

### BACKGROUND

Respiration is one of the four vital signs of the human body, and it is a necessary process of gas exchange between the internal and external environment of the human body. Human respiratory activity need to be realized by the cooperation of several systems such as respiration, nerve and movement system. In order to better reflect the characteristics of human respiratory activity, it is necessary to convert human respiratory activity into the form of respiratory signal, so as to observe and understand the situation of human respiratory activity. However, if the respiratory signal is obtained by using a non-contact piezoelectric sensing method, the obtained piezoelectric sensing signal includes not only the respiratory signal, but also a lot of noise interference, resulting in a very low signal-to-noise ratio of the obtained respiratory signal.

### SUMMARY

To overcome the problems existing in related technologies, embodiments of the present disclosure provide a method, device and computer system for obtaining respiratory signal.

According to a first aspect of an embodiment of the present disclosure, a method for obtaining respiratory signal is provided, which includes the following steps:
obtaining aliasing vital signs signals of a target human body through a piezoelectric sensor; the aliasing vital signs signals include a respiratory signal and other noise signals;
based on the aliasing vital signs signals obtained by the piezoelectric sensor, performing the following steps through a processor:
filtering the aliasing vital signs signals to obtain target vital signs signals including the respiratory signal;
performing a Fourier transform on the target vital signs signals to obtain first frequency response in preset frequency range; and generating an upper envelope according to each frequency point of the first frequency response and a preset value range; the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point;
in the first frequency response, obtaining the frequency point corresponding to the maximum value of the flat tops; determining it as main peak frequency point, and determining the amplitude corresponding to the main peak frequency point as main peak amplitude;
in the upper envelope, identifying the flat top corresponding to the main peak amplitude as main peak flat top; in the first frequency response, determining minimum value frequency point according to the minimum value between the main peak flat top and an adjacent flat top or flat bottom, and determining respiratory spectrum principal component interval according to the minimum value frequency point; the number of the minimum value frequency points is 1-2; when there is only one minimum value frequency point, the starting frequency point of the corresponding respiratory spectrum principal component interval is 0, and the ending frequency point is the only one minimum value frequency point; when there are two minimum value frequency points, the corresponding respiratory spectrum principal component interval is the range between the two minimum value frequency points;
reconstructing the respiratory spectrum principal component interval through an empirical wavelet function to obtain reconstructed respiratory signal.

In an alternative embodiment, wherein the step of obtaining first frequency response and generating an upper envelope according to each frequency point of the first frequency response and a preset value range includes:
obtaining a plurality of local maximum frequency points according to local maximum values of a plurality of preset local ranges on the first frequency response, and obtaining minimum interval of adjacent local maximum frequency points;
obtaining the value range according to the spectral resolution of the first frequency response and the minimum interval;
determining amplitude of the upper envelope corresponding to each frequency point as the maximum amplitude of the frequency point within the value range corresponding to each frequency point;
generating the upper envelope according to the amplitude of the upper envelope corresponding to all the frequency points.

In an alternative embodiment, wherein before the step of identifying the flat top corresponding to the main peak amplitude as main peak flat top in the upper envelope; determining minimum value frequency point according to the minimum value between the main peak flat top and an adjacent flat top or flat bottom in the first frequency response, and determining respiratory spectrum principal component interval according to the minimum value frequency point, includes:
in the first frequency response, obtaining the frequency point corresponding to the second maximum value of the flat tops; determining the frequency point as sub peak frequency point, and determining the amplitude corresponding to the sub peak frequency point as sub peak amplitude;
comparing the sub peak amplitude and the main peak amplitude after preset amplitude reduction; if the sub peak amplitude greater than or equal to the main peak amplitude after preset amplitude reduction, performing a short time Fourier transform on the target vital signs signals to obtain a plurality of second frequency responses in preset frequency range; wherein the duration corresponding to each second frequency response is less than the duration corresponding to the first frequency response;
obtaining the statistical number of the sub frequency responses that the main peak amplitude and the sub peak amplitude both exist and the sub peak amplitude is greater than or equal to the main peak amplitude after preset amplitude reduction;
if the statistical number is greater than the half of total number of the second frequency responses, replacing the original main peak frequency point and the main peak amplitude with the sub peak frequency point and the sub peak amplitude to become new main peak frequency point and new main peak amplitude.

In an alternative embodiment, wherein the method for obtaining respiratory signal includes:
if the statistical number is less than or equal to the half of total number of the second frequency responses, splitting the time domain of the target vital signs signals to obtain two segments of vital signs sub-signals including the main peak frequency point and the main peak amplitude and the sub peak frequency point and the sub peak amplitude respectively;
determining the vital signs sub-signals as the new target vital signs signals, and re-executing the step of performing a Fourier transform on the target vital signs signals to obtain first frequency response in preset frequency range; and generating an upper envelope according to each frequency point of the first frequency response and a preset value range.

In an alternative embodiment, wherein the minimum value frequency points include a first minimum value frequency point and a second minimum value frequency point respectively located on both sides of the main peak frequency point;
the step of reconstructing the respiratory spectrum principal component interval through an empirical wavelet function to obtain reconstructed respiratory signal, comprises:
obtaining a first minimum value frequency point and a second minimum value frequency point in the respiratory spectrum principal component interval;
reconstructing the respiratory spectrum principal component interval through the following formula:

$$ψ ( f ) = \{ \begin{matrix}1 , & ( 1 + γ ) {f}_{L} \leq f \leq ( 1 - γ ) {f}_{H} \\ cos [ \frac{π}{2} β ( \frac{1}{2 γ {f}_{H}} ( f - ( 1 - γ ) {f}_{H} ) ) ] , & ( 1 - γ ) {f}_{H} \leq f \leq ( 1 + γ ) {f}_{H} \\ sin [ \frac{π}{2} β ( \frac{1}{2 γ {f}_{L}} ( f - ( 1 - γ ) {f}_{L} ) ) ] , & ( 1 - γ ) {f}_{H} \leq f \leq ( + - γ ) {f}_{L} \\ 0 , & otherwise\end{matrix} ;$$

wherein, β(x)=x⁴(35−84x+70x²−20x³), ψ(f) is the output of the empirical wavelet function, represents the reconstructed respiratory signal; γ is a preset coefficient; f_{L} represents the first minimum value frequency point; f_{H} represents the second minimum value frequency point.

According to a second aspect of an embodiment of the present disclosure, a device for obtaining respiratory signal is provided, which includes:
a piezoelectric sensor, configured to obtain aliasing vital signs signals of the target human body; the aliasing vital signs signals include a respiratory signal and other noise signals;
a processor, and a plurality of modules executed by the processor, the plurality of modules comprises:
filter processing module, configured to filter the aliasing vital signs signals to obtain target vital signs signals including the respiratory signal;
upper envelope generates module, configured to perform a Fourier transform on the target vital signs signals to obtain first frequency response in preset frequency range; and generate an upper envelope according to each frequency point of the first frequency response and a preset value range; the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point;
main peak acquisition module, configured to obtain the frequency point corresponding to the maximum value of the flat tops in the first frequency response; determine it as main peak frequency point, and determine the amplitude corresponding to the main peak frequency point as main peak amplitude;
respiratory spectrum principal component interval acquisition module, configured to identify the flat top corresponding to the main peak amplitude as main peak flat top in the upper envelope; determine minimum value frequency point according to the minimum value between the main peak flat top and an adjacent flat top or flat bottom in the first frequency response, and determine respiratory spectrum principal component interval according to the minimum value frequency point;
respiratory signal reconstruction module, configured to reconstruct the respiratory spectrum principal component interval through an empirical wavelet function to obtain reconstructed respiratory signal.

According to a third aspect of an embodiment of the present disclosure, a computer system for obtaining respiratory signal is provided, which includes:
a processor;
a memory; and
a computer program stored in the memory and executable by the processor, wherein the processor executes the computer program to implement the steps of the method for obtaining respiratory signal of the first aspect of an embodiment of the present disclosure.

Compared with prior art, the disclosure obtains the aliasing vital signs signals of the target human body through the piezoelectric sensor, and then filters out most noise interference and retains the target vital signs signals including the respiratory signal, and then generates the first frequency response corresponding to the target vital signs signals through Fourier transform. Based on the first frequency response, generating the upper envelope, and obtaining the respiratory spectrum principal component interval through the flat tops and flat bottoms of the upper envelope. Finally, reconstructing the respiratory spectrum principal component interval through the empirical wavelet function to obtain reconstructed respiratory signal. The disclosure solves the problem of strong binding caused by direct contact with the test object when collecting the signal of the existing medical device and wearable product and can reconstitute the signal of the respiratory spectrum principal component interval through the empirical wavelet function in the mixed aliasing vital signs signals to extract the accurate respiratory signal, so as to improve the accuracy of the obtained respiratory signal.

It should be understood that the above general description and the subsequent detailed description are only illustrative and explanatory but not limiting to the present disclosure.

For a better understanding and implementation, the disclosure will be described in detail below in combination with the accompanying drawings.

### BRIEF DESCRIPTION OF THE DRAWINGS

FIG. 1 is a schematic hardware diagram of the device for obtaining respiratory signal provided by the present disclosure;

FIG. 2 is a schematic flow diagram of the method for obtaining respiratory signal executed by the processor;

FIG. 3 is a schematic diagram of the upper envelope of an embodiment of the disclosure;

FIG. 4 is a schematic flow diagram of the step of S3 of the method for obtaining respiratory signal provided by an embodiment of the disclosure;

FIG. 5 is a schematic module diagram of the device for obtaining respiratory signal provided by the present disclosure;

FIG. 6 is a schematic structural diagram of a computer system provided by an embodiment of the present disclosure.

### DETAILED DESCRIPTION

In order to make the purpose, technical scheme and advantages of the disclosure more clear, the embodiments of the disclosure will be further described in detail in combination with the attached drawings.

It should be made clear that the embodiments described are only a portion of the embodiments of the disclosure and not the entirety of the embodiments. Based on the embodiments of the disclosure, all other embodiments obtained by a person of ordinary skill in the field without creative labor fall within the scope of protection of the embodiments of this application.

Where the description below relates to drawings, the same numbers in different drawings represent the same or similar elements unless otherwise indicated. In the description of the disclosure, it is to be understood that the terms “first”, “second”, “third”, etc., are used only to distinguish similar objects and are not necessarily used to describe a particular order or precedence, nor can they be understood to indicate or imply relative importance. For a person of ordinary skill in the field, the specific meaning of the above terms in the disclosure may be understood on a case-by-case basis. The terms “a” and “the” in the singular form used in the disclosure and the accompanying claims are also intended to include the majority form, unless the context clearly indicates otherwise. The words “if”' used here can be interpreted as “in . . . ”, “when . . . ” or “respond to a determination that . . . ”.

In addition, in the description of the disclosure, “multiple” means two or more unless otherwise stated. “And/or” describes the association relationship of the associated object, indicating that there can be three kinds of relationships, for example, A and/or B, can be: A exists alone, A and B exist simultaneously, and B exists alone. The character “/” generally indicates that the associated object is an “or” relationship.

The above embodiments are only used to illustrate the implementation of the present disclosure, but not to limit it. While the present application has been described with reference to above embodiments in detail, it will be understood by those skilled in the art that various changes may be made and equivalents may be substituted for elements thereof without departing from the scope of the present disclosure.

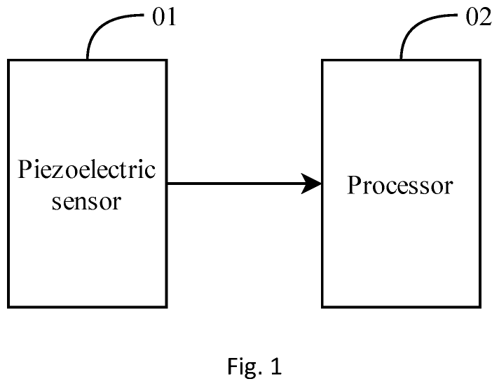

Referring to FIG. 1, which is a schematic hardware diagram of a device for obtaining respiratory signal provided by the present disclosure. The device for obtaining respiratory signal provided by an embodiment of the present disclosure includes:

A piezoelectric sensor 01, configured to obtain aliasing vital signs signals of a target human body; the aliasing vital signs signals include a respiratory signal and other noise signals.

A processor 02, configured to perform a method for obtaining respiratory signal provided by the present disclosure based on the aliasing vital signs signals obtained by the piezoelectric sensor 01.

The piezoelectric sensor is a detecting device, which can sense the measured information; The perceived information can be transformed into an electrical signal or other required form of information output according to a certain law. The piezoelectric sensor can be placed inside a mattress or pillow.

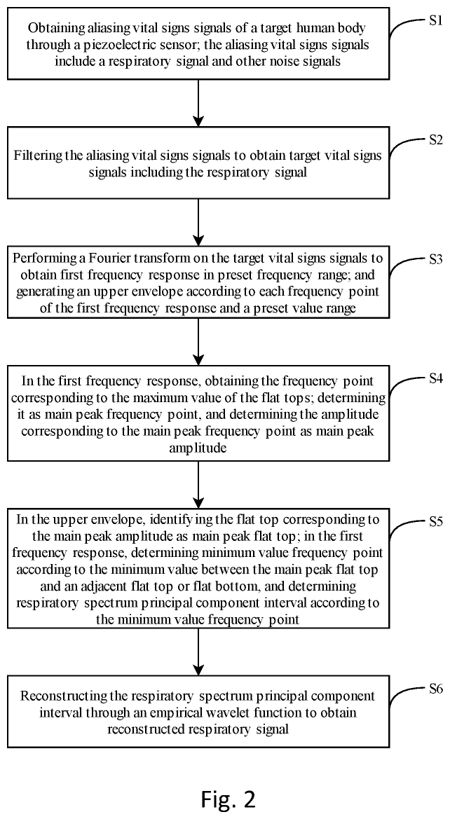

Referring to FIG. 2, which is a schematic flow diagram of the method for obtaining respiratory signal executed by the processor 02. The method for obtaining respiratory signal provided by an embodiment of the present disclosure includes:
S1, obtaining the aliasing vital signs signals of the target human body collected by the piezoelectric sensor 01; the aliasing vital signs signals include a respiratory signal and other noise signals.

The respiratory signal is obtained by converting the breathing state of the human body into a signal form through the piezoelectric sensor 01. The respiratory signal embodies breath-related parameters such as respiratory rhythm and respiratory effort. Wherein, the respiratory rhythm refers to the speed of breathing, and the respiratory effort refers to the depth of breathing.

The other noise signals refer to signals other than respiratory signal obtained by the piezoelectric sensor 01. These signals are caused by the body's heartbeat, movements, and external influences.
S2, filtering the aliasing vital signs signals to obtain target vital signs signals including the respiratory signal.

The filtering processing includes but is not limited to notch filtering and low-pass filtering. The notch filtering can suppress the influence of power frequency interference on the target vital signs signals. The low-pass filtering suppress the influence of Gaussian noise on the target vital signs signals.

Specifically, in an embodiment, the step of S2 includes: filtering out body motion interference signals caused by the physical activities of the target human body through a preset body motion recognition algorithm; filtering out 50 Hz power frequency interference through a second-order notch filter; filtering out Gaussian noise and some vital signs signals unrelated to the respiratory signals through a second-order Butterworth low-pass filter with a cutoff frequency of 1 Hz. The reason why the selection cutoff frequency of the second-order Butterworth low-pass filter is set to 1 Hz is that under conventional conditions, the cycle frequency of the respiratory signal does not exceed 60 times/minute, so the frequency of the respiratory signal under conventional conditions is less than or equal to 1 Hz. However, in other embodiments, the cutoff frequency of the second-order Butterworth low-pass filter may be appropriately enlarged to avoid the inability to acquire respiratory signals with frequency exceeding 60 times/min.
S3, performing a Fourier transform on the target vital signs signals to obtain first frequency response in preset frequency range; and generating an upper envelope according to each frequency point of the first frequency response and a preset value range; the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point.

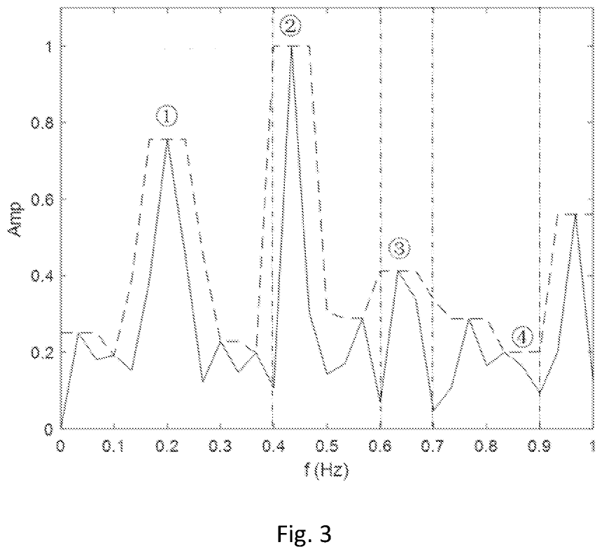

Referring to FIG. 3, which is a schematic diagram of the upper envelope of an embodiment of the disclosure. In the upper envelope, the flat tops have a “convex” shape, and the flat bottoms have a “concave” shape. Therefore, the flat tops and flat bottoms of the upper envelope can be judged by the following mathematical formula:

Condition 1: If the amplitude of the upper envelope corresponding to multiple consecutive frequency points is the same, the multiple frequency points are determined as a same frequency interval, and jump to condition 2;

Condition 2: The amplitude of the upper envelope corresponding to the two frequency points adjacent to the frequency interval is determined as the amplitude of adjacent upper envelope line; If the amplitude of two adjacent upper envelope lines is greater than the amplitude of the upper envelope corresponding to the frequency interval, the frequency interval is determined as a flat bottom; If the amplitude of two adjacent upper envelope lines is smaller than the amplitude of the upper envelope corresponding to the frequency interval, the frequency interval is determined as a flat top.
S4, In the first frequency response, obtaining the frequency point corresponding to the maximum value of the flat tops; determining it as main peak frequency point, and determining the amplitude corresponding to the main peak frequency point as main peak amplitude.
S5, In the upper envelope, identifying the flat top corresponding to the main peak amplitude as main peak flat top; in the amplitude-frequency response, determining minimum value frequency point according to the minimum value between the main peak flat top and an adjacent flat top or flat bottom, and determining respiratory spectrum principal component interval according to the minimum value frequency point.

The number of the minimum value frequency points is 1-2. For example, when there is neither a flat top nor a flat bottom on the left side of the main peak flat top, there is only one minimum value frequency point located on the right side of the main peak flat top, and the starting frequency point of the corresponding respiratory spectrum principal component interval is 0, and the ending frequency point is the only one minimum value frequency point. When there are flat tops and flat bottoms on the left and right sides of the main peak flat top, there is a minimum value frequency point respectively on the left and right sides of the main peak flat top, and the corresponding respiratory spectrum principal component interval is the range between the two minimum value frequency points.
S6, reconstructing the respiratory spectrum principal component interval through an empirical wavelet function to obtain reconstructed respiratory signal.

Compared with prior art, the disclosure obtains the aliasing vital signs signals of the target human body through the piezoelectric sensor 01, and then filters out most noise interference and retains the target vital signs signals including the respiratory signal, and then generates the first frequency response corresponding to the target vital signs signals through Fourier transform. Based on the first frequency response, generating the upper envelope, and obtaining the respiratory spectrum principal component interval through the flat tops and flat bottoms of the upper envelope. Finally, reconstructing the respiratory spectrum principal component interval through the empirical wavelet function to obtain reconstructed respiratory signal. The disclosure solves the problem of strong binding caused by direct contact with the test object when collecting the signal of the existing medical device and wearable product, and can reconstitute the signal of the respiratory spectrum principal component interval through the empirical wavelet function in the mixed aliasing vital signs signals to extract the accurate respiratory signal, so as to improve the accuracy of the obtained respiratory signal.

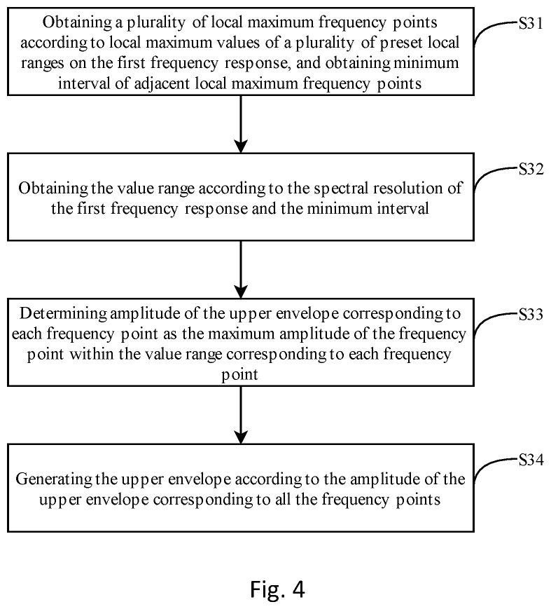

Referring to FIG. 4, which is a schematic flow diagram of the step of S3 of the method for obtaining respiratory signal provided by an embodiment of the present disclosure. In an embodiment, wherein the step of S3, performing a Fourier transform on the target vital signs signals to obtain first frequency response in preset frequency range and generating an upper envelope according to each frequency point of the first frequency response and a preset value range, include:
S31, obtaining a plurality of local maximum frequency points according to local maximum values of a plurality of preset local ranges on the first frequency response, and obtaining minimum interval of adjacent local maximum frequency points.

The minimum interval refers to the smallest of the interval distances of each adjacent local maximum frequency points.
S32, obtaining the value range according to the spectral resolution of the first frequency response and the minimum interval.
S33, determining amplitude of the upper envelope corresponding to each frequency point as the maximum amplitude of the frequency point within the value range corresponding to each frequency point.
S34, generating the upper envelope according to the amplitude of the upper envelope corresponding to all the frequency points.

For example, the value range can be expressed as:

XU(f)=max{[X(f−f min+Δf), X(f+f min−Δf)]};

Wherein, XU(f) represents the amplitude of the upper envelope corresponding to the present frequency point; f represents the present frequency point; f min represents the minimum interval; Δf represents the spectral resolution of the first frequency response.

In the present embodiment, obtaining the value range according to the spectral resolution of the first frequency response and the minimum interval to generate the upper envelope, thereby improving the accuracy of the generated upper envelope.

In a feasible embodiment, before the step of S5 of identifying the flat top

corresponding to the main peak amplitude as main peak flat top in the upper envelope; determining minimum value frequency point according to the minimum value between the main peak flat top and an adjacent flat top or flat bottom in the first frequency response, and determining respiratory spectrum principal component interval according to the minimum value frequency point, includes:
in the first frequency response, obtaining the frequency point corresponding to the second maximum value of the flat tops; determining the frequency point as sub peak frequency point, and determining the amplitude corresponding to the sub peak frequency point as sub peak amplitude;
comparing the sub peak amplitude and the main peak amplitude after preset amplitude reduction; if the sub peak amplitude greater than or equal to the main peak amplitude after preset amplitude reduction, performing a short time Fourier transform on the target vital signs signals to obtain a plurality of second frequency responses in preset frequency range; wherein the duration corresponding to each second frequency response is less than the duration corresponding to the first frequency response;
obtaining the statistical number of the sub frequency responses that the main peak amplitude and the sub peak amplitude both exist and the sub peak amplitude is greater than or equal to the main peak amplitude after preset amplitude reduction;
if the statistical number is greater than the half of total number of the second frequency responses, replacing the original main peak frequency point and the main peak amplitude with the sub peak frequency point and the sub peak amplitude to become new main peak frequency point and new main peak amplitude.

According to the comparison between the sub peak amplitude and the main peak amplitude in each second frequency response, determining whether the main peak frequency point and the main peak amplitude need to be updated to improve the positioning accuracy of the main peak.

In the present embodiment, if the statistical number is less than or equal to the half of total number of the second frequency responses, splitting the time domain of the target vital signs signals to obtain two segments of vital signs sub-signals including the main peak frequency point and the main peak amplitude and the sub peak frequency point and the sub peak amplitude respectively;
determining the vital signs sub-signals as the new target vital signs signals, and re-executing the step of performing a Fourier transform on the target vital signs signals to obtain first frequency response in preset frequency range; and generating an upper envelope according to each frequency point of the first frequency response and a preset value range.

In the present embodiment, considering that there may be complex breaths with multiple respiratory rates and similar durations over a period of time, result in there are two breathing rates in the first frequency response; therefore, according to the comparison between the sub peak amplitude and the main peak amplitude in each second frequency response, determining whether the main peak frequency point and the sub peak frequency point need to be divided into two segments of target vital signs signals to obtain their respiratory signal respectively, so as to improve the accuracy of the respiratory spectrum principal component interval obtained by each segment of the target vital signs signals.

In a feasible embodiment, the minimum value frequency points include a first minimum value frequency point and a second minimum value frequency point respectively located on both sides of the main peak frequency point;

The step of S6 of reconstructing the respiratory spectrum principal component interval through an empirical wavelet function to obtain reconstructed respiratory signal, includes:
obtaining a first minimum value frequency point and a second minimum value frequency point in the respiratory spectrum principal component interval;
reconstructing the respiratory spectrum principal component interval through the following formula:

$$ψ ( f ) = \{ \begin{matrix}1 , & ( 1 + γ ) {f}_{L} \leq f \leq ( 1 - γ ) {f}_{H} \\ cos [ \frac{π}{2} β ( \frac{1}{2 γ {f}_{H}} ( f - ( 1 - γ ) {f}_{H} ) ) ] , & ( 1 - γ ) {f}_{H} \leq f \leq ( 1 + γ ) {f}_{H} \\ sin [ \frac{π}{2} β ( \frac{1}{2 γ {f}_{L}} ( f - ( 1 - γ ) {f}_{L} ) ) ] , & ( 1 - γ ) {f}_{H} \leq f \leq ( 1 + γ ) {f}_{L} \\ 0 , & otherwise\end{matrix} ;$$

wherein, β(x)=x⁴(35−84x+70x²−20x³); ψ(f) is the output of the empirical wavelet function, represents the reconstructed respiratory signal; γ is a preset coefficient; f_{L} represents the first minimum value frequency point; f_{H} represents the second minimum value frequency point.

In the present embodiment, reconstructing the respiratory spectrum principal component interval according to the empirical wavelet function, the first minimum value frequency point and the second minimum value frequency point in the respiratory spectrum principal component interval, so as to improve the accuracy of the acquired respiratory signal. However, if there is neither a flat top nor a flat bottom on the left side of the main peak flat top, there is only one minimum value frequency point located on the right side of the main peak flat top; in the above case, determining 0 Hz in the first frequency response as the first minimum value frequency point, and determining the only one minimum value frequency point as the second minimum value frequency point.

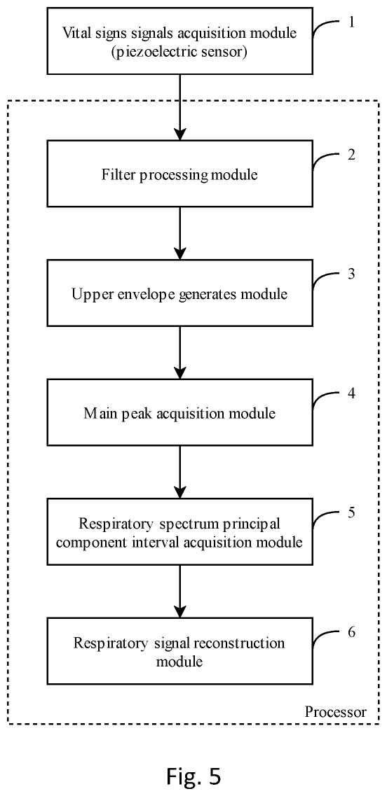

Referring to FIG. 5, which is a schematic module diagram of a device for obtaining respiratory signal provided by the present disclosure. The device for obtaining respiratory signal provided by an embodiment of the present disclosure includes :

Vital signs signals acquisition module 1, configured to obtain aliasing vital signs signals of the target human body; the aliasing vital signs signals include a respiratory signal and other noise signals; the vital signs signals acquisition module 1 is a piezoelectric sensor.

The piezoelectric sensor is a detecting device, which can sense the measured information; The perceived information can be transformed into an electrical signal or other required form of information output according to a certain law. The piezoelectric sensor can be placed inside a mattress or pillow.

A processor, and a plurality of modules executed by the processor, the plurality of modules include:
filter processing module 2, configured to filter the aliasing vital signs signals to obtain target vital signs signals including the respiratory signal;
upper envelope generates module 3, configured to perform a Fourier transform on the target vital signs signals to obtain first frequency response in preset frequency range; and generate an upper envelope according to each frequency point of the first frequency response and a preset value range; the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point;
main peak acquisition module 4, configured to obtain the frequency point corresponding to the maximum value of the flat tops in the first frequency response; determine it as main peak frequency point, and determine the amplitude corresponding to the main peak frequency point as main peak amplitude;
respiratory spectrum principal component interval acquisition module 5, configured to identify the flat top corresponding to the main peak amplitude as main peak flat top in the upper envelope; determine minimum value frequency point according to the minimum value between the main peak flat top and an adjacent flat top or flat bottom in the first frequency response, and determine respiratory spectrum principal component interval according to the minimum value frequency point;
respiratory signal reconstruction module 6, configured to reconstruct the respiratory spectrum principal component interval through an empirical wavelet function to obtain reconstructed respiratory signal.

The respiratory signal is obtained by converting the breathing state of the human body into a signal form through the piezoelectric sensor 01. The respiratory signal embodies breath-related parameters such as respiratory rhythm and respiratory effort. Wherein, the respiratory rhythm refers to the speed of breathing, and the respiratory effort refers to the depth of breathing.

The other noise signals refer to signals other than respiratory signal obtained by the piezoelectric sensor 01. These signals are caused by the body's heartbeat, movements, and external influences.

The filtering processing includes but is not limited to notch filtering and low-pass filtering. The notch filtering can suppress the influence of power frequency interference on the target vital signs signals. The low-pass filtering suppress the influence of Gaussian noise on the target vital signs signals.

Specifically, in an embodiment, the step which the filter processing module 2 preforms, includes: filtering out body motion interference signals caused by the physical activities of the target human body through a preset body motion recognition algorithm; filtering out 50 Hz power frequency interference through a second-order notch filter;

filtering out Gaussian noise and some vital signs signals unrelated to the respiratory signals through a second-order Butterworth low-pass filter with a cutoff frequency of 1 Hz. The reason why the selection cutoff frequency of the second-order Butterworth low-pass filter is set to 1 Hz is that under conventional conditions, the cycle frequency of the respiratory signal does not exceed 60 times/minute, so the frequency of the respiratory signal under conventional conditions is less than or equal to 1 Hz. However, in other embodiments, the cutoff frequency of the second-order Butterworth low-pass filter may be appropriately enlarged to avoid the inability to acquire respiratory signals with frequency exceeding 60 times/min.

In the upper envelope, the flat tops have a “convex” shape, and the flat bottoms have a “concave” shape. Therefore, the flat tops and flat bottoms of the upper envelope can be judged by the following mathematical formula:

Condition 1: If the amplitude of the upper envelope corresponding to multiple consecutive frequency points is the same, the multiple frequency points are determined as a same frequency interval, and jump to condition 2;

Condition 2: The amplitude of the upper envelope corresponding to the two frequency points adjacent to the frequency interval is determined as the amplitude of adjacent upper envelope line; If the amplitude of two adjacent upper envelope lines is greater than the amplitude of the upper envelope corresponding to the frequency interval, the frequency interval is determined as a flat bottom; If the amplitude of two adjacent upper envelope lines is smaller than the amplitude of the upper envelope corresponding to the frequency interval, the frequency interval is determined as a flat top.

The number of the minimum value frequency points is 1-2. For example, when there is neither a flat top nor a flat bottom on the left side of the main peak flat top, there is only one minimum value frequency point located on the right side of the main peak flat top, and the starting frequency point of the corresponding respiratory spectrum principal component interval is 0, and the ending frequency point is the smallest value frequency point. When there are flat tops and flat bottoms on the left and right sides of the main peak flat top, there is a minimum value frequency point respectively on the left and right sides of the main peak flat top, and the corresponding respiratory spectrum principal component interval is the range between the two minimum value frequency points.

Compared with prior art, the disclosure obtains the aliasing vital signs signals of the target human body through the piezoelectric sensor 01, and then filters out most noise interference and retains the target vital signs signals including the respiratory signal, and then generates the first frequency response corresponding to the target vital signs signals through Fourier transform. Based on the first frequency response, generating the upper envelope, and obtaining the respiratory spectrum principal component interval through the flat tops and flat bottoms of the upper envelope. Finally, reconstructing the respiratory spectrum principal component interval through the empirical wavelet function to obtain reconstructed respiratory signal. The disclosure solves the problem of strong binding caused by direct contact with the test object when collecting the signal of the existing medical device and wearable product, and can reconstitute the signal of the respiratory spectrum principal component interval through the empirical wavelet function in the mixed aliasing vital signs signals to extract the accurate respiratory signal, so as to improve the accuracy of the obtained respiratory signal.

In a feasible embodiment, the upper envelope generates module 3 includes the following sub-modules:
minimum interval acquisition sub-module 31, configured to obtain a plurality of local maximum frequency points according to local maximum values of a plurality of preset local ranges on the first frequency response, and obtain minimum interval of adjacent local maximum frequency points;
value range acquisition sub-module 32, configured to obtain the value range according to the spectral resolution of the first frequency response and the minimum interval;
amplitude of upper envelope acquisition sub-module 33, configured to determine amplitude of the upper envelope corresponding to each frequency point as the maximum amplitude of the frequency point within the value range corresponding to each frequency point;
upper envelope generation sub-module 34, configured to generate the upper envelope according to the amplitude of the upper envelope corresponding to all the frequency points.

The minimum interval refers to the smallest of the interval distances of each adjacent local maximum frequency points.

In the embodiment, obtaining the value range according to the spectral resolution of the first frequency response and the minimum interval, to generate the corresponding upper envelope, so as to improve the accuracy of the generated upper envelope.

In a feasible embodiment, the respiratory spectrum principal component interval acquisition module 5 includes the following sub-modules:
sub peak frequency point acquisition sub-module 51, configured to obtain the frequency point corresponding to the second maximum value of the flat tops in the first frequency response; determine the frequency point as sub peak frequency point, and determine the amplitude corresponding to the sub peak frequency point as sub peak amplitude;
second frequency response acquisition sub-module 52, configured to compare the sub peak amplitude and the main peak amplitude after preset amplitude reduction; if the sub peak amplitude greater than or equal to the main peak amplitude after preset amplitude reduction, perform a short time Fourier transform on the target vital signs signals to obtain a plurality of second frequency responses in preset frequency range; wherein the duration corresponding to each second frequency response is less than the duration corresponding to the first frequency response;
second frequency response statistics sub-module 53, configured to obtain the statistical number of the sub frequency responses that the main peak amplitude and the sub peak amplitude both exist and the sub peak amplitude is greater than or equal to the main peak amplitude after preset amplitude reduction;
main peak update sub-module 54, configured to replace the original main peak frequency point and the main peak amplitude with the sub peak frequency point if the statistical number is greater than the half of total number of the second frequency responses, and the sub peak amplitude to become new main peak frequency point and new main peak amplitude.

In the embodiment, according to the comparison between the sub peak amplitude and the main peak amplitude in each second frequency response, determining whether the main peak frequency point and the main peak amplitude need to be updated to improve the positioning accuracy of the main peak.

In a feasible embodiment, the respiratory spectrum principal component interval acquisition module 5, also includes:
time domain segmentation sub-module 55, configured to if the statistical number is less than or equal to the half of total number of the second frequency responses, split the time domain of the target vital signs signals to obtain two segments of vital signs sub-signals including the main peak frequency point and the main peak amplitude and the sub peak frequency point and the sub peak amplitude respectively;
the upper envelope generates module 3, also configured to determine the vital signs sub-signals as the new target vital signs signals, re-execute the step of performing a Fourier transform on the target vital signs signals to obtain first frequency response in preset frequency range; and generate an upper envelope according to each frequency point of the first frequency response and a preset value range.

In the embodiment, according to the comparison between the sub peak amplitude and the main peak amplitude in each second frequency response, determining whether the main peak frequency point and the sub peak frequency point need to be divided into two segments of target vital signs signals to obtain their respiratory signal respectively, so as to improve the accuracy of the respiratory spectrum principal component interval obtained by each segment of the target vital signs signals.

The disclosure also provides application modes based on the method for obtaining respiratory signal, including:

### (1) Determination of Respiratory Rhythm

Obtaining target respiratory signal according to a plurality of the reconstructed respiratory signals obtained by the method for obtaining respiratory signal.

Calculating the total number of peaks and valleys of the target respiratory signal in a preset duration through the following formula:

PV=30(Nₚ+N_{y})/T;

Wherein, PV represents the total number of peaks and valleys of the target respiratory signal in a preset duration; T represents the duration of the target respiratory signal; Nₚ represents the number of peaks of the target respiratory signal; N_{y} represents the number of valleys of the target respiratory signal.

Determining respiratory rhythm level of the target respiratory signal according to the total number of peaks and valleys:
if the total number of peaks and valleys is less than a preset first threshold of peaks and valleys, determining that the respiratory rhythm of the target respiratory signal is lower than normal respiratory rhythm;
if the total number of peaks and valleys is greater than or equal to the preset first threshold of peaks and valleys, and is less than a preset second threshold of peaks and valleys, determining that the respiratory rhythm of the target respiratory signal is the normal respiratory rhythm;
if the total number of peaks and valleys is greater than or equal to the preset second threshold of peaks and valleys, determining that the respiratory rhythm of the target respiratory signal is higher than the normal respiratory rhythm.

In an embodiment, the first threshold of peaks and valleys is 9 and the second threshold of peaks and valleys is 18.

### (2) Determination of Respiratory Effort

Obtaining target respiratory signal according to a plurality of the reconstructed respiratory signals obtained by the method for obtaining respiratory signal.

Calculating the variance of the target respiratory signal.

Determining respiratory effort level of the target respiratory signal according to the variance:
if the variance is less than a preset first threshold of variance, determining that the respiratory effort is lower than normal respiratory effort;
if the variance is greater than or equal to the preset first threshold of variance, and is less than a preset second threshold of variance, determining that the respiratory effort is the normal respiratory effort;
if the variance is greater than or equal to the second threshold of variance, determining that the respiratory effort is higher than the normal respiratory effort.
In an embodiment, the first threshold of variance is 25, and the second threshold of variance is 100. In other embodiments, users can also make a more accurate judgment of the respiratory effort of the target respiratory signal through other thresholds of variance.

### (3) Determination of Respiratory Disturbance

Obtaining target respiratory signal according to a plurality of the reconstructed respiratory signals obtained by the method for obtaining respiratory signal.

Calculating interval standard deviation of respiratory interval of the target respiratory signal.

Determining respiratory disturbance of the target respiratory signal according to the interval standard deviation: if the interval standard deviation is greater or equal to a preset threshold of interval, determining that the target respiratory signal presents a respiratory disorder.

In an embodiment, the preset threshold of interval is 0.85.

### (4) Determination of Sleep Apnea Hypopnea

Obtaining target respiratory signal according to a plurality of the reconstructed respiratory signals obtained by the method for obtaining respiratory signal.

Calculating the kurtosis of the target respiratory signal.

Determining sleep apnea hypopnea of the target respiratory signal according to the kurtosis. For example, if the kurtosis is greater than or equal to a preset first threshold of kurtosis, the target respiratory signal is suspected to be central sleep apnea; if the kurtosis is less than the first threshold of kurtosis, and is greater than or equal to a preset second threshold of kurtosis, the target respiratory signal is suspected to be mixed sleep apnea; if the kurtosis is less than the second threshold of kurtosis, and is greater than or equal to a preset third threshold of kurtosis, the target respiratory signal is suspected to present as obstructive sleep apnea or hypopnea. Wherein, in an embodiment, the first threshold of kurtosis is 3; the second threshold of kurtosis is 2.5; the third threshold of kurtosis is 2.

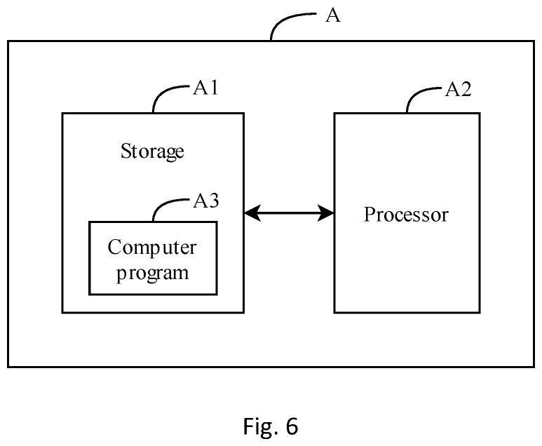

Referring to FIG. 6, which is a schematic structural diagram of a computer system provided by an embodiment of the present disclosure. An embodiment of the disclosure also provides a computer system A, including a storage A1, a processor A2, and a computer program A3 stored in the storage A1 that can be executed by the processor A2. When the processor A2 executes the computer program A3, it implements the method for obtaining respiratory signal described above.

The embodiments described above are schematic only, where the components described as separate components may or may not be physically separate, and the components shown as units may or may not be physical units, that is, may be located in one place, or may be distributed over multiple network units. Part or all of the modules can be selected according to the actual needs to achieve the purpose of the scheme provided by the disclosure. It can be understood and implemented by ordinary skilled persons in the field without creative labor.

Skilled persons in the field should understand that embodiments of the disclosure may be provided as methods, systems, or computer program products. Therefore, the disclosure may take the form of a full hardware embodiment, a full software embodiment, or a combination of software and hardware embodiments. Further, the disclosure may take the form of a computer program product implemented on one or more computer available storage media (including but not limited to disk memory, CD-ROM, optical memory, etc.) which contain computer available program code.

The disclosure is described by reference to flow charts and/or block diagrams of methods, equipment (systems), and computer program products in accordance with the disclosure. It should be understood that each process and/or box in a flowchart and/or block diagram, and a combination of processes and/or boxes in a flowchart and/or block diagram, can be implemented by a computer program instruction. These computer program instructions may be supplied to a general-purpose computer, a special purpose computer, an embedded processor, or a processor of other programmable data processing equipment to produce a machine. Causes instructions executed by the processor of a computer or other programmable data processing device to produce a device used to implement a selected function in a flow diagram or flow diagrams and/or block diagrams in a box or boxes. These computer program instructions may also be stored in computer-readable memory capable of directing a computer or other programmable data processing device to work in a particular manner such that the instructions stored in such computer-readable memory produce manufactured goods including instruction devices. The instruction device implements selected functions in a flowchart for one process or more processes and/or a block diagram for one box or more boxes.

These computer program instructions may also be loaded onto a computer or other programmable data processing device, such that a series of operational steps are performed on the computer or other programmable data processing device to produce computer-implemented processing. Instructions thus executed on a computer or other programmable device provide steps for implementing a function selected in a flowchart for a process or processes and/or a block diagram for a box or boxes.

In a typical configuration, a computing device includes one or more processors (CPU), input/output interfaces, network interfaces, and memory.

Memory may include non-permanent memory in computer readable media, random access memory (RAM) and/or non-volatile memory in the form of read-only memory (ROM) or flash memory (flash RAM). Memory is an example of a computer readable medium.

Computer readable media, including permanent and non-permanent, removable and non-removable media, can be stored by any method or technology. Information can be computer-readable instructions, data structures, modules of programs, or other data. Examples of computer storage media include, But not limited to phase change memory (PRAM), static random access memory (SRAM), dynamic random access memory (DRAM), other types of random access memory (RAM), read-only memory (ROM), electrically erasable programmable Read only memory (EEPROM), flash memory or other memory technologies, CD-ROM Read only memory (ROM) A CD-ROM, digital multifunctional disc (DVD) or other optical storage, magnetic cassette, magnetic disk storage or other magnetic storage device or any other non-transmission medium that may be used to store information that can be accessed by a computing device. As defined herein, computer-readable media does not include transitory media, such as modulated data signals and carriers.

It should also be noted that the term “includes”, “comprises” or any other variation thereof is intended to cover non-exclusive inclusion, so that a process, method, good or equipment comprising a set of elements includes not only those elements but also other elements not expressly listed or that are inherent to the process, method, good or equipment. In the absence of further restrictions, the sentence “including a . . . ” A defined element does not preclude the existence of additional identical elements in the process, method, product or equipment comprising the element.

The above are examples of the disclosure only and are not intended to limit the disclosure. For persons skilled in the art, the disclosure is subject to various changes and variations. Any modification, equivalent substitution, improvement, etc. made within the spirit and principle of the disclosure shall be included in the claims of the disclosure.

What is claimed:

1. A method for obtaining respiratory signal, comprises:
obtaining aliasing vital signs signals of a target human body through a piezoelectric sensor; the aliasing vital signs signals include a respiratory signal and other noise signals;
based on the aliasing vital signs signals obtained by the piezoelectric sensor, performing the following steps through a processor:
filtering the aliasing vital signs signals to obtain target vital signs signals including the respiratory signal;
performing a Fourier transform on the target vital signs signals to obtain first frequency response in preset frequency range; and generating an upper envelope according to each frequency point of the first frequency response and a preset value range;
the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point;
in the first frequency response, obtaining the frequency point corresponding to the maximum value of the flat tops; determining it as main peak frequency point, and determining the amplitude corresponding to the main peak frequency point as main peak amplitude;
in the upper envelope, identifying the flat top corresponding to the main peak amplitude as main peak flat top; in the first frequency response, determining minimum value frequency point according to the minimum value between the main peak flat top and an adjacent flat top or flat bottom, and determining respiratory spectrum principal component interval according to the minimum value frequency point; the number of the minimum value frequency points is 1-2; when there is only one minimum value frequency point, the starting frequency point of the corresponding respiratory spectrum principal component interval is 0, and the ending frequency point is the only one minimum value frequency point; when there are two minimum value frequency points, the corresponding respiratory spectrum principal component interval is the range between the two minimum value frequency points;
reconstructing the respiratory spectrum principal component interval through an empirical wavelet function to obtain reconstructed respiratory signal.

2. The method for obtaining respiratory signal of claim 1, wherein the step of obtaining first frequency response and generating an upper envelope according to each frequency point of the first frequency response and a preset value range comprises:
obtaining a plurality of local maximum frequency points according to local maximum values of a plurality of preset local ranges on the first frequency response, and obtaining minimum interval of adjacent local maximum frequency points;
obtaining the value range according to the spectral resolution of the first frequency response and the minimum interval;
determining amplitude of the upper envelope corresponding to each frequency point as the maximum amplitude of the frequency point within the value range corresponding to each frequency point;
generating the upper envelope according to the amplitude of the upper envelope corresponding to all the frequency points.

3. The method for obtaining respiratory signal of claim 1, wherein before the step of identifying the flat top corresponding to the main peak amplitude as main peak flat top in the upper envelope; determining minimum value frequency point according to the minimum value between the main peak flat top and an adjacent flat top or flat bottom in the first frequency response, and determining respiratory spectrum principal component interval according to the minimum value frequency point, comprises:
in the first frequency response, obtaining the frequency point corresponding to the second maximum value of the flat tops; determining the frequency point as sub peak frequency point, and determining the amplitude corresponding to the sub peak frequency point as sub peak amplitude;
comparing the sub peak amplitude and the main peak amplitude after preset amplitude reduction; if the sub peak amplitude greater than or equal to the main peak amplitude after preset amplitude reduction, performing a short time Fourier transform on the target vital signs signals to obtain a plurality of second frequency responses in preset frequency range; wherein the duration corresponding to each second frequency response is less than the duration corresponding to the first frequency response;
obtaining the statistical number of the sub frequency responses that the main peak amplitude and the sub peak amplitude both exist and the sub peak amplitude is greater than or equal to the main peak amplitude after preset amplitude reduction;
if the statistical number is greater than the half of total number of the second frequency responses, replacing the original main peak frequency point and the main peak amplitude with the sub peak frequency point and the sub peak amplitude to become new main peak frequency point and new main peak amplitude.

4. The method for obtaining respiratory signal of claim 3, comprises:
if the statistical number is less than or equal to the half of total number of the second frequency responses, splitting the time domain of the target vital signs signals to obtain two segments of vital signs sub-signals including the main peak frequency point and the main peak amplitude and the sub peak frequency point and the sub peak amplitude respectively;
determining the vital signs sub-signals as the new target vital signs signals, and re-executing the step of performing a Fourier transform on the target vital signs signals to obtain first frequency response in preset frequency range; and generating an upper envelope according to each frequency point of the first frequency response and a preset value range.

5. The method for obtaining respiratory signal of claim 1, wherein the minimum value frequency points include a first minimum value frequency point and a second minimum value frequency point respectively located on both sides of the main peak frequency point;
the step of reconstructing the respiratory spectrum principal component interval through an empirical wavelet function to obtain reconstructed respiratory signal, comprises:
obtaining a first minimum value frequency point and a second minimum value frequency point in the respiratory spectrum principal component interval;
reconstructing the respiratory spectrum principal component interval through the following formula:
$$ψ ( f ) = \{ \begin{matrix}1 , & ( 1 + γ ) {f}_{L} \leq f \leq ( 1 - γ ) {f}_{H} \\ cos [ \frac{π}{2} β ( \frac{1}{2 γ {f}_{H}} ( f - ( 1 - γ ) {f}_{H} ) ) ] , & ( 1 - γ ) {f}_{H} \leq f \leq ( 1 + γ ) {f}_{H} \\ sin [ \frac{π}{2} β ( \frac{1}{2 γ {f}_{L}} ( f - ( 1 - γ ) {f}_{L} ) ) ] , & ( 1 - γ ) {f}_{H} \leq f \leq ( 1 + γ ) {f}_{L} \\ 0 , & otherwise\end{matrix} ;$$
wherein, β(x)=x⁴(35−84x+70x²−20x³); ψ(f) is the output of the empirical wavelet function, represents the reconstructed respiratory signal; γ is a preset coefficient; f_{L} represents the first minimum value frequency point; f_{H} represents the second minimum value frequency point.

6. A device for obtaining respiratory signal, comprises:
a piezoelectric sensor, configured to obtain aliasing vital signs signals of the target human body; the aliasing vital signs signals include a respiratory signal and other noise signals;
a processor, and a plurality of modules executed by the processor, the plurality of modules comprises:
filter processing module, configured to filter the aliasing vital signs signals to obtain target vital signs signals including the respiratory signal;
upper envelope generates module, configured to perform a Fourier transform on the target vital signs signals to obtain first frequency response in preset frequency range; and generate an upper envelope according to each frequency point of the first frequency response and a preset value range; the upper envelope includes a plurality of flat tops and flat bottoms, wherein the amplitude of the frequency point of the flat top is greater than the amplitude of the adjacent non-flat top frequency point, and the amplitude of the frequency point of the flat bottom is less than the amplitude of the adjacent non-flat bottom frequency point;
main peak acquisition module, configured to obtain the frequency point corresponding to the maximum value of the flat tops in the first frequency response; determine it as main peak frequency point, and determine the amplitude corresponding to the main peak frequency point as main peak amplitude;
respiratory spectrum principal component interval acquisition module, configured to identify the flat top corresponding to the main peak amplitude as main peak flat top in the upper envelope; determine minimum value frequency point according to the minimum value between the main peak flat top and an adjacent flat top or flat bottom in the first frequency response, and determine respiratory spectrum principal component interval according to the minimum value frequency point;
respiratory signal reconstruction module, configured to reconstruct the respiratory spectrum principal component interval through an empirical wavelet function to obtain reconstructed respiratory signal.

7. The device for obtaining respiratory signal of claim 6, wherein the upper envelope generates module comprises the following sub-modules:
minimum interval acquisition sub-module, configured to obtain a plurality of local maximum frequency points according to local maximum values of a plurality of preset local ranges on the first frequency response, and obtain minimum interval of adjacent local maximum frequency points;
value range acquisition sub-module, configured to obtain the value range according to the spectral resolution of the first frequency response and the minimum interval;
amplitude of upper envelope acquisition sub-module, configured to determine amplitude of the upper envelope corresponding to each frequency point as the maximum amplitude of the frequency point within the value range corresponding to each frequency point;
upper envelope generation sub-module, configured to generate the upper envelope according to the amplitude of the upper envelope corresponding to all the frequency points.

8. The device for obtaining respiratory signal of claim 6, wherein the respiratory spectrum principal component interval acquisition module comprises the following sub-modules:
sub peak frequency point acquisition sub-module, configured to obtain the frequency point corresponding to the second maximum value of the flat tops in the first frequency response; determine the frequency point as sub peak frequency point, and determine the amplitude corresponding to the sub peak frequency point as sub peak amplitude;
second frequency response acquisition sub-module, configured to compare the sub peak amplitude and the main peak amplitude after preset amplitude reduction; if the sub peak amplitude greater than or equal to the main peak amplitude after preset amplitude reduction, perform a short time Fourier transform on the target vital signs signals to obtain a plurality of second frequency responses in preset frequency range; wherein the duration corresponding to each second frequency response is less than the duration corresponding to the first frequency response;
second frequency response statistics sub-module, configured to obtain the statistical number of the sub frequency responses that the main peak amplitude and the sub peak amplitude both exist and the sub peak amplitude is greater than or equal to the main peak amplitude after preset amplitude reduction;
main peak update sub-module, configured to replace the original main peak frequency point and the main peak amplitude with the sub peak frequency point if the statistical number is greater than the half of total number of the second frequency responses, and the sub peak amplitude to become new main peak frequency point and new main peak amplitude.

9. The device for obtaining respiratory signal of claim 8, wherein the respiratory spectrum principal component interval acquisition module, comprises:
time domain segmentation sub-module, configured to if the statistical number is less than or equal to the half of total number of the second frequency responses, split the time domain of the target vital signs signals to obtain two segments of vital signs sub-signals including the main peak frequency point and the main peak amplitude and the sub peak frequency point and the sub peak amplitude respectively;
the upper envelope generates module, also configured to determine the vital signs sub-signals as the new target vital signs signals, re-execute the step of performing a Fourier transform on the target vital signs signals to obtain first frequency response in preset frequency range; and generate an upper envelope according to each frequency point of the first frequency response and a preset value range.

10. The device for obtaining respiratory signal of claim 6, wherein the minimum value frequency points include a first minimum value frequency point and a second minimum value frequency point respectively located on both sides of the main peak frequency point;
the step executed by the respiratory signal reconstruction module of reconstructing the respiratory spectrum principal component interval through an empirical wavelet function to obtain reconstructed respiratory signal, comprises:
obtaining a first minimum value frequency point and a second minimum value frequency point in the respiratory spectrum principal component interval;
reconstructing the respiratory spectrum principal component interval through the following formula:
$$ψ ( f ) = \{ \begin{matrix}1 , & ( 1 + γ ) {f}_{L} \leq f \leq ( 1 - γ ) {f}_{H} \\ cos [ \frac{π}{2} β ( \frac{1}{2 γ {f}_{H}} ( f - ( 1 - γ ) {f}_{H} ) ) ] , & ( 1 - γ ) {f}_{H} \leq f \leq ( 1 + γ ) {f}_{H} \\ sin [ \frac{π}{2} β ( \frac{1}{2 γ {f}_{L}} ( f - ( 1 - γ ) {f}_{L} ) ) ] , & ( 1 - γ ) {f}_{H} \leq f \leq ( 1 + γ ) {f}_{L} \\ 0 , & otherwise\end{matrix} ;$$
wherein, β(x)=x⁴(35−84x+70x²−20x³); ψ(f) is the output of the empirical wavelet function, represents the reconstructed respiratory signal; γ is a preset coefficient; f_{L} represents the first minimum value frequency point; f_{H} represents the second minimum value frequency point.

11. A computer system for obtaining respiratory signal, comprising:
a processor;
a memory; and
a computer program stored in the memory and executable by the processor, wherein the processor executes the computer program to implement the steps of the method for obtaining respiratory signal of claim 1.

12. The computer system for obtaining respiratory signal of claim 11, wherein the method that is implemented by the processor comprises:
wherein the step of obtaining first frequency response and generating an upper envelope according to each frequency point of the first frequency response and a preset value range comprises:
obtaining a plurality of local maximum frequency points according to local maximum values of a plurality of preset local ranges on the first frequency response, and obtaining minimum interval of adjacent local maximum frequency points;
obtaining the value range according to the spectral resolution of the first frequency response and the minimum interval;
determining amplitude of the upper envelope corresponding to each frequency point as the maximum amplitude of the frequency point within the value range corresponding to each frequency point;
generating the upper envelope according to the amplitude of the upper envelope corresponding to all the frequency points.

13. The computer system for obtaining respiratory signal of claim 11, wherein the method that is implemented by the processor comprises:
wherein before the step of identifying the flat top corresponding to the main peak amplitude as main peak flat top in the upper envelope; determining minimum value frequency point according to the minimum value between the main peak flat top and an adjacent flat top or flat bottom in the amplitude-frequency response, and determining respiratory spectrum principal component interval according to the minimum value frequency point, comprises:
in the first frequency response, obtaining the frequency point corresponding to the second maximum value of the flat tops; determining the frequency point as sub peak frequency point, and determining the amplitude corresponding to the sub peak frequency point as sub peak amplitude;
comparing the sub peak amplitude and the main peak amplitude after preset amplitude reduction; if the sub peak amplitude greater than or equal to the main peak amplitude after preset amplitude reduction, performing a short time Fourier transform on the target vital signs signals to obtain a plurality of second frequency responses in preset frequency range; wherein the duration corresponding to each second frequency response is less than the duration corresponding to the first frequency response;
obtaining the statistical number of the sub frequency responses that the main peak amplitude and the sub peak amplitude both exist and the sub peak amplitude is greater than or equal to the main peak amplitude after preset amplitude reduction;
if the statistical number is greater than the half of total number of the second frequency responses, replacing the original main peak frequency point and the main peak amplitude with the sub peak frequency point and the sub peak amplitude to become new main peak frequency point and new main peak amplitude.

14. The computer system for obtaining respiratory signal of claim 13, wherein the method that is implemented by the processor comprises:
if the statistical number is less than or equal to the half of total number of the second frequency responses, splitting the time domain of the target vital signs signals to obtain two segments of vital signs sub-signals including the main peak frequency point and the main peak amplitude and the sub peak frequency point and the sub peak amplitude respectively;
determining the vital signs sub-signals as the new target vital signs signals, and re-executing the step of performing a Fourier transform on the target vital signs signals to obtain first frequency response in preset frequency range; and generating an upper envelope according to each frequency point of the first frequency response and a preset value range.

15. The computer system for obtaining respiratory signal of claim 11, wherein the method that is implemented by the processor comprises:
wherein the minimum value frequency points include a first minimum value frequency point and a second minimum value frequency point respectively located on both sides of the main peak frequency point;
the step of reconstructing the respiratory spectrum principal component interval through an empirical wavelet function to obtain reconstructed respiratory signal, comprises:
obtaining a first minimum value frequency point and a second minimum value frequency point in the respiratory spectrum principal component interval;
reconstructing the respiratory spectrum principal component interval through the following formula:
$$ψ ( f ) = \{ \begin{matrix}1 , & ( 1 + γ ) {f}_{L} \leq f \leq ( 1 - γ ) {f}_{H} \\ cos [ \frac{π}{2} β ( \frac{1}{2 γ {f}_{H}} ( f - ( 1 - γ ) {f}_{H} ) ) ] , & ( 1 - γ ) {f}_{H} \leq f \leq ( 1 + γ ) {f}_{H} \\ sin [ \frac{π}{2} β ( \frac{1}{2 γ {f}_{L}} ( f - ( 1 - γ ) {f}_{L} ) ) ] , & ( 1 - γ ) {f}_{H} \leq f \leq ( 1 + γ ) {f}_{L} \\ 0 , & otherwise\end{matrix} ;$$
wherein, β(x)=x⁴(35−84x+70x²−20x³); ψ(f) is the output of the empirical wavelet function, represents the reconstructed respiratory signal; γ is a preset coefficient; f_{L} represents the first minimum value frequency point; f_{H} represents the second minimum value frequency point.
